(19) United States
(12) Patent Application Publication
(10) Pub. No.: US 2023/0355726 A1
(43) Pub. Date: Nov. 9, 2023

(54) FIBRINOLYTIC ENZYME COMPOSITION FOR FIBROSIS TREATMENT

(71) Applicant: Advanced Enzyme Technologies Ltd., Maharashtra (IN)

(72) Inventors: Vasant Laxminarayan RATHI, Yorba Linda, CA (US); Neha SHAH, Fullerton, CA (US)

(73) Assignee: Advanced Enzyme Technologies Ltd., Maharashtra (IN)

(21) Appl. No.: 17/847,753

(22) Filed: Jun. 23, 2022

(30) Foreign Application Priority Data
May 6, 2022 (IN) 202121003637

(51) Int. Cl. A61K 38/54; A61K 38/48

(52) U.S. Cl. CPC A61K 38/54; A61K 38/482; A61K 38/4886; A61K 38/4873; C12Y 304/2404; C12Y 304/21062; C12Y 304/22033

(57) ABSTRACT

An oral fibrinolytic composition comprising the enzymes serrapeptase and nattokinase. The composition treats and/or may prevent fibrosis conditions and their related symptoms in animals, including humans. This composition may further comprise of one or more enzymes, bioflavonoids, vitamins, coenzymes, minerals, probiotics, prebiotics, herbs, excipients and/or combinations thereof. The composition may improve lung capacity, oxygen saturation, vigor, and/or lung function. It may further alleviate dyspnea, limitations and fear caused by dyspnea, cough, phlegm, fatigue, body pain, chest discomfort, anxiety, depression, loss of appetite, and/or respiratory disorders. The composition of serrapeptase and nattokinase and/or one or more additional component may improve immune health, sleep, health-related quality of life (HRQoL), social well-being, health status, mental health and/or exercise capacity. The present disclosure also provides a method for improving or reducing the frequency and severity of fibrosis and related symptoms in a subject having a fibrotic disease.

### CROSS-REFERENCE TO RELATED APPLICATIONS

This disclosure claims priority to Indian Patent Application No. 202121003637 filed on May 6, 2022 and titled “FIBRINOLYTIC ENZYME COMPOSITION FOR FIBROSIS TREATMENT”, which is incorporated by reference in its entirety.

### TECHNICAL FIELD

The present disclosure relates to a composition for treatment of fibrosis in animals, including humans. The present disclosure also relates to a method for improving fibrosis and related symptoms or reducing the frequency and severity of fibrosis and related symptoms in a subject having a fibrotic disease.

### BACKGROUND

Fibrosis can occur because of several pathogenic mechanisms including chronic inflammation, abnormal wound healing after tissue injury, oxidative stress and procoagulant mechanisms. Fibrosis is characterized by an accumulation of excess extracellular matrix (ECM) components. In the body, there is a balance between ECM deposition and breakdown. In fibrosing conditions such as pulmonary fibrosis, rheumatoid arthritis, scleroderma, systemic lupus erythematosus etc., this balance is shifted towards more deposition which eventually leads to a loss of tissue function. A “hyperactive” wound healing response also results in fibrosis and prolonged inflammation. This process can occur in tissues such as skin, liver, kidney, heart and lungs following surgery, mechanical or chemical injury.

Pulmonary fibrosis is a disease in which the lung tissues become fibrotic. Patients diagnosed with pulmonary fibrosis suffer from a build-up of fibrin in the interstitial tissue of the lungs which causes the lung tissue to thicken and become stiff.

When a person is diagnosed with pulmonary fibrosis, a doctor may be able to determine the etiology of the illness. In many circumstances, a doctor may be unable to determine why a person has developed pulmonary fibrosis. Fibrosis is referred to as “idiopathic” when cause of the disease is unknown.

Cases of unknown-cause, or idiopathic pulmonary fibrosis (IPF), are particularly difficult to resolve. IPF is a progressive disease with a high mortality rate, if left untreated. Pulmonary hypertension and respiratory failure are two other IPF problems that occur when the lungs are unable to carry enough oxygen into the bloodstream without assistance. IPF is a life-threatening condition that results in respiratory failure and death.

Pulmonary fibrosis patients suffer from poor quality of life primarily because of the burden of symptoms such as dyspnea and cough. Progressive activity-limiting dyspnea is one of the main symptoms of IPF and is a major concern of all pulmonary fibrosis patients.

Pulmonary fibrosis patients also frequently experience fear, anxiety, worry, hopelessness and helplessness and accordingly have an impact on physical, social and emotional well-being.

There are very few treatment options for pulmonary fibrosis available today. Although available antifibrotic therapies slow down disease progression, they have no impact on existing scarring. Lung scarring once developed, is permanent and there is no evidence of any available medication that can reverse this condition. Further, the available medications have been associated with side effects such as appetite loss, nausea, diarrhea, lethargy and photosensitivity. Moreover, available antifibrotic therapies do not meaningfully impact quality of life. Therefore, it is important to treat the disease and its symptoms along with other aspects of patient care and wellbeing.

Accordingly, there is a need to develop novel treatments that improve fibrosis, its symptoms and quality of life in patients with fibrotic conditions.

### SUMMARY

In one embodiment, a composition comprising the enzymes serrapeptase and nattokinase, and/or one or more additional components for the treatment of fibrosis is disclosed.

In another embodiment, a composition comprising the enzymes serrapeptase and nattokinase, and/or one or more additional components for improving/reducing the frequency and severity of fibrosis and related symptoms in a subject having a fibrotic disease is disclosed.

In still another embodiment, a composition comprising the enzymes serrapeptase and nattokinase, and/or one or more additional components in treatment of idiopathic pulmonary fibrosis is disclosed.

### BRIEF DESCRIPTION OF THE DRAWINGS

Reference will be made to embodiments of the invention, examples of which may be illustrated in accompanying figures. These figures are intended to be illustrative, not limiting. Although the invention is generally described in the context of these embodiments, the disclosure is not intended to limit the scope of the invention to these particular embodiments.

FIG. 1 shows the effect of the supplements Serracor-NK and Serra Rx260 on WHO (five) well-being index (1998 version) scores in subjects with pulmonary fibrosis (End of Study scores as compared to Baseline).

FIG. 2 shows the effect of the supplements Serracor-NK and Serra Rx260 on University of California San Diego Shortness-of-Breath (UCSD-SOB) scores in subjects with pulmonary fibrosis (End of Study scores as compared to Baseline).

FIG. 3 shows the effect of the supplements Serracor-NK and Serra Rx260 on St. George's Respiratory Questionnaire (SGRQ) Total, Symptoms, Activity and Impact scores in subjects with pulmonary fibrosis (End of Study scores as compared to Baseline).

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

Aspects of the present invention is directed towards a composition for treatment of fibrosis. Use of this composition may improve fibrosis and its symptoms in patients with fibrotic conditions, as well as the patients' ability to perform the activities of daily living and their mental and physical wellbeing, thus enhancing their overall quality of life.

The composition for treatment of fibrosis comprises of serrapeptase and nattokinase, and/or one or more additional components.

Serrapeptase is a zinc containing metalloprotease of molecular weight 45-60 KDa. It is a proteolytic enzyme that specifically catalyzes the cleaving of proteins into smaller components. Serrapeptase was originally discovered in the intestine of the silkworm Bombyx mori. When a silkworm evolves into a moth, it employs serrapeptase to dissolve its cocoon. It is now typically isolated from the bacteria Serratia marcescens. Serrapeptase activity is usually measured in SPU. One unit of serrapeptase activity, or one SPU, yields a product equivalent to 1.0 microgram of tyrosine per minute at pH 9.0 and 37 degrees Celsius on casein substrate. Other common names for serrapeptase are serralysin, serratiapeptase, serratia peptidase, serratiopeptidase, serrapeptidase, etc.

Nattokinase is a serine protease of molecular weight ˜27 KDa. It is a proteolytic enzyme that specifically catalyzes the cleaving of proteins into smaller components. Nattokinase is commonly found in fermented soybeans known as “natto.” It is now typically isolated from the bacteria Bacillus subtilis. Nattokinase activity is usually measured in FU. One unit of nattokinase activity, or one FU, is defined as the amount of enzyme which increases the absorbance of the filtrate at 275 nm by 0.01 per minute at pH 8.5 and 37 degree Celsius on fibrin substrate.

In an embodiment, the additional component is selected from one or more enzymes, bioflavonoids, vitamins, coenzymes, minerals, herbs, antioxidants, carotenoids, probiotics, prebiotics, or mixtures thereof.

Suitable enzymes can be selected from, but not limited to, bromelain, papain, lipases, proteases, glucose oxidase, amylase, carbohydrase, cellulase, mannanase, pectinase and mixtures thereof.

Suitable bioflavonoids can be selected from, but not limited to, rutin quercetin, silibinin, silymarin, puerarin, baicalin, baicalein, hesperidin, genistein, naringenin, hydroxysafflor yellow A, oroxylinA, anthocyanins, kaempferol breviscapine, galangin, and skullcapflavone I.

Suitable vitamins can be selected from, but not limited to fat soluble vitamins (vitamin A, D, E, K) and water-soluble vitamins (vitamin C and B complex).

Suitable coenzymes can be selected from, but not limited to, nicotinamide adenine dinucleotide, flavin adenine dinucleotide, Coenzyme A, and Coenzyme Q10.

Suitable minerals can be selected from, but not limited to, sodium, chloride, potassium, calcium, phosphorus, magnesium, sulfur, iron, zinc, iodine, selenium, copper, manganese, fluoride, chromium, and molybdenum.

Suitable herbs can be selected from, but not limited to families of Phyllanthaceae (Amla), Euphorbiaceae, Fabaceae, Asteraceae, Lamiaceae, Matricaria chamomilla, Echinacea purpurea, Allium sativum, Zingiber officinale, Ginkgo biloba, Panax ginseng, and Curcumin longa.

Suitable carotenoids can be selected from, but not limited to, beta carotene, lycopene, lutein, and zeaxanthin.

Suitable probiotics can be selected from, but not limited to, genera Lactobacillus, Bifidobacterium, Saccharomyces, Enterococcus, Streptococcus, Pediococcus, Leuconostoc, Bacillus, and Escherichia coli.

Suitable prebiotics can be selected from, but not limited to, fructooligosaccharides, glucan, galactooligosaccharides, maltooligosaccharides, isomaltulose, xylooligosaccharides, cyclodextrins, raffinose oligosaccharides, lactulose, lactosucrose, palatinose, isomaltooligosaccharides, and arabinoxyloligosaccharides.

In another embodiment of the invention stabilizers and diluents are selected from maltodextrin, microcrystalline cellulose, and other suitable diluents from edible sources, including, but not limited to, corn, tapioca, potato, rice wheat.

In another embodiment of the invention the composition comprises serrapeptase, nattokinase, bromelain, papain, lipases, proteases, rutin, amla, coenzyme Q10, magnesium and mixtures thereof for treatment of fibrosis.

In an embodiment, the serrapeptase used to formulate the composition has an activity ranging from 100 SPU/g to 2 million SPU/g.

In an embodiment, the nattokinase used to formulate the composition has an activity ranging from 50 FU/g to 20,000 FU/g.

In an embodiment, the composition has an activity ranging from 500 FU/g to 50,000 FU/g

In an embodiment, the one or more additional component is present in an amount range of 1 mg to 950 mg per gram of the composition.

In an embodiment, bromelain is an additional component in the formulation. Bromelain is a proteolytic enzyme typically derived from the Bromeliaceae family of plants (Pineapple). Bromelain activity is usually measured in gelatin digestion unit (GDU). One GDU is that amount of enzyme which will liberate, after 20 minutes digestion at 45 degrees Celsius, 1 milligram of amino nitrogen from a standard gelatin at pH 4.5. In this embodiment, the bromelain used to formulate the composition has an activity ranging from 30 GDU/g to 3000 GDU/g.

In another embodiment of the invention, the serrapeptase and nattokinase and/or one or more additional components thereof are protected with a substance that delays the release of active components from immediately releasing in the stomach. These coatings are often referred to as “enteric coatings.”

In an embodiment, the enteric coatings are applied to serrapeptase, nattokinase or a mixture thereof because these ingredients are more sensitive to denaturation in an acidic pH. Such enteric coating is applied to protect them from the acidic pH of the stomach, to prevent gastric distress, and to deliver active enzymes to the absorption site.

In another embodiment, the enteric coatings are applied to the oral delivery system (e.g., tablet or capsule or similar).

Suitable enteric coatings can be selected from pH dependent coatings as well as pH independent coatings including, but not limited to, cellulose acetate phthalate, cellulose acetate trimellitate, hydroxyl propyl methyl cellulose phthalate, hydroxyl propyl methyl cellulose acetate succinate, polyvinyl acetate phthalate, methacrylic acid copolymer, carrageenan, and corn proteins.

Another embodiment of the invention relates to the use of a composition comprising serrapeptase, nattokinase and/or one or more additional components in treating fibrosis.

In an embodiment, the fibrosis is idiopathic pulmonary fibrosis.

In an embodiment, the invention relates to the use of composition comprising serrapeptase, and nattokinase and/or one or more additional components for improving the health status, lung function, sleep cycle, health related quality of life (HRQoL), mental and physical well-being, social well-being, and/or exercise capacity in a subject having a fibrotic disease.

Yet another embodiment of the invention relates to the use of composition comprising serrapeptase, and nattokinase and/or one or more additional components for improving fibrosis and related symptoms and/or reducing the frequency and severity of fibrosis and related symptoms in a subject having a fibrotic disease

Yet another embodiment of the invention relates to the use of composition comprising serrapeptase, and nattokinase and/or one or more additional components for oxygen saturation, vigor, lung function, cough, phlegm, fatigue, body pain, chest discomfort, anxiety, depression, loss of appetite, respiratory disorders and/or alleviating dyspnea, exertional dyspnea, limitations and fear caused by dyspnea or combinations in a subject having a fibrotic disease.

Yet another embodiment of the invention relates to the use of composition comprising serrapeptase, and nattokinase and/or one or more additional component for improving the immune health by combating the oxidative stress and inflammation in animals, including humans. Oxidative stress results from excess production of reactive oxygen species (ROS) or a depletion of antioxidant defenses and causes molecular, cellular and tissue abnormalities. Oxidative stress has been implicated in pulmonary fibrosis, as well as fibrosis in other organs such as the liver and heart. Uncontrolled activation of the coagulation cascade following tissue injury leads to inflammation and fibrosis and can contribute to fibrosis of the lung, liver, kidney and heart. Therefore, fibrosis can affect nearly every tissue in the body and is a major cause of morbidity and mortality.

Yet another embodiment of the invention relates to the use of composition comprising serrapeptase, and nattokinase and/or one or more additional component for components for the treatment of Pyeronie's syndrome, which is a condition caused by fibrous scar tissue forming in the penis, causing it to become curved and painful.

In an embodiment, the composition comprising serrapeptase, and nattokinase and/or one or more additional component is administered in a single dosage form containing about 400 to 600 mg of composition, and in one embodiment, 500 mg of the composition.

In an embodiment, the composition comprising serrapeptase, and nattokinase and/or one or more additional component may be administered to the patient in one or more doses per 12 hours, and in one embodiment, from 1 to 5 doses per 12 hours.

In an embodiment, the composition comprises serrapeptase, and nattokinase and/or one or more additional component is administered in a single dosage form and is administered orally, preferably in the form of a tablet, capsule, powder, effervescent, liquid solution, or liquid suspension.

In an embodiment of the invention serrapeptase and nattokinase owing to their fibrinolytic, anti-inflammatory and immunomodulatory effects are beneficial in limiting and reducing the amount of scar tissue and thus alleviating symptoms.

In an embodiment of the invention, the proteolytic enzymes, serrapeptase, nattokinase have fibrinolytic in vitro and in silico activity. Since factors involved in the pathogenesis of PF include chronic inflammation, an uncontrolled healing response and progressive fibrosis or scarring, treatment with the systemic enzymes serrapeptase and nattokinase could be achieved.

In an embodiment of the present invention the enzyme composition for treatment of fibrosis may comprise pharmaceutically acceptable excipients.

Suitable pharmaceutically acceptable excipients can be selected from, such as but not limited to maltodextrin, microcrystalline cellulose, starches, sugars, lactose, and gelatin. The pharmaceutically acceptable excipients, as described herein, can be used alone or in combination.

### EXAMPLE

The following example is illustrative of the invention but not limitative of the scope thereof:

### Materials and Methods

Material: Serracor-NK is a blend of enzymes, antioxidants, bioflavonoids, essential vitamins and minerals, including serrapeptase, nattokinase, bromelain, papain, lipase, rutin, amla, coenzyme Q10 and magnesium.

Ethical considerations: The study was conducted as per the ethical principles contained in the current revision of the “Declaration of Helsinki 2013”, ICH harmonized guideline integrated addendum to ICH E6(R1): Guidelines for Good Clinical Practice ICH E6(R2) and following all applicable laws and regulations. No vulnerable subject participated in the study. The study was approved by the Argus IRB, Tuscon, Arizona in October 2019.

Inclusion criteria: Subjects who provided written informed consent; subjects with a diagnosis of Pulmonary Fibrosis; and aged ≥18 years were included in the study.

Exclusion Criteria: Subjects on any anti-coagulant medications, e.g., Aspirin, Plavix, or Brilinta; subjects who have taken enzyme supplements within the past 30 days before start of study treatment; allergy or sensitivity to enzyme supplements; subjects that are not able to read English, understand the questionnaires or follow study procedures; and subjects who are pregnant or planning to become pregnant were considered ineligible to participate in the study.

Study Design: Single arm, open label study, examining the effects of the systemic enzyme supplements Serracor-NK+Serra Rx on symptoms and quality of life in patients with pulmonary fibrosis.

A total of 13 subjects were enrolled between November 2019 and August 2020.

The subjects received the oral supplements Serracor-NK and Serra Rx three times a day for 12 weeks. Subjects completed three quality of life questionnaires—the World Health Organization-5 Well-Being Index (WHO-5), the University of California San Diego-Shortness of Breath Questionnaire (UCSD-SOB) and the Saint George's Respiratory Questionnaire (SGRQ), at baseline and at various time points.

Dose: Table 1 provides the dosing schedule for the supplements.

Subjects received 1 capsule of Serracor-NK 3 times a day on days 1 to 4. They received 2 capsules of Serracor-NK 3 times a day on days 5 to 8. Subjects received a therapeutic dose of 2 capsules of Serracor-NK and 1 capsule of Serra Rx 3 times a day on days 9 to 92. The supplements were taken on an empty stomach (1 hour before or 2 hours after a meal) with a cup of water.

TABLE 1
Dosing Schedule
Directions: TAKE ON AN EMPTY STOMACH (45 minutes
before a meal or 2 hours after a meal, with a glass of water)

| | |
|---|---|
| Step 1 | Take 1 capsule Serracor-NK, 3 times a day |
| (Days 1-4) | *Due to the body not being accustomed to systemic enzyme |
| | therapy, it is important to start at a minimal dosage and |
| | increase as needed. Minor symptoms of intestinal cleansing |
| | may occur |
| Step 2 | Take 2 capsules Serracor-NK, 3 times a day |
| (Days 5-8) | *At this point, your body should be completed with the |
| | cleansing stage and becoming more accustomed to systemic |
| | enzyme therapy. |
| Step 3 | Take 2 capsules Serracor-NK + 1 capsule Serra Rx, 3 |
| (Days 9-92) | times a day |
| | *This is what we believe to be the therapeutic dosage |

Screening and enrolment: After obtaining informed consent, subjects were screened as mentioned in the study calendar (Table 2). After confirmation of eligibility, participants were enrolled in the study. All subjects received the supplements and assessments were performed as mentioned in the study calendar (Table 2). Day 100 was considered the end of the study period.

TABLE 2
Study Calendar

| | | | Days from | | | |
|---|---|---|---|---|---|---|
| REQUIRED | Screening | Day 0 | Registration | | | |
| STUDY | (Day-14 | (Baseline/ | Day | Day | Day | Day |
| PROCEDURE | to Day-1) | Registration) | 36 | 64 | 92 | 100 |
| Informed | ✓ | | | | | |
| Consent Fonn | | | | | | |
| HIPAA | ✓ | | | | | |
| Authorization | | | | | | |
| Intake Form | ✓ | | | | | |
| (demographics, | | | | | | |
| disease severity, | | | | | | |
| comorbidities) | | | | | | |
| UCSD-SOB | | ✓ | ✓ | ✓ | ✓ | |
| (approx. 7 | | | | | | |
| minutes to | | | | | | |
| complete) | | | | | | |
| SGRQ (approx. | | ✓ | ✓ | ✓ | ✓ | |
| 9 minutes to | | | | | | |
| complete) | | | | | | |
| WHO-5 (approx. | | ✓ | ✓ | ✓ | ✓ | |
| 2 minutes to | | | | | | |
| complete) | | | | | | |

| | |
|---|---|
| Supplement Intake* | Daily, starting day 1 |
| Medication Log | Daily, starting day 1 |
| Follow-up | |

*Follow the dosing schedule (Table 1) for supplement intake

Statistical analysis: The categorical variables were expressed as frequencies and percentages. The scoring and percentage of subjects showing improvement as compared to baseline using the study instruments was calculated as follows:

WHO (Five) Well-Being Index (WHO-5): The WHO-5 is a short self-reported measure of current mental well-being. It consists of 5 positively framed statements which subjects rate from 0 (none of the time) to 5 (all of the time). A raw score of 0 represents worst possible quality of life (QOL) and 25 represents best possible QOL. Percentage score was calculated by multiplying the raw score by 4. A change in score of 10% is considered significant.

The University of California San Diego—Shortness of breath questionnaire (UCSD-SOB): This is a 24-item measure that assess self-reported shortness of breath while performing a variety of activities of daily living (ADL). The severity of SOB is rated from 0 (none at all) to 5 (maximum or unable to do because of shortness of breath) in 21 ADL. Three additional questions ask about fear of harm from overexertion, limitations and fear caused by SOB. Scores range from 0 to 120, with higher scores indicating greater dyspnea. A change of 5 units is considered a reasonable minimal clinically important difference for this instrument.

Saint George's Respiratory questionnaire (SGRQ): This is a standardized self-administered airways disease-specific 50 item questionnaire split into three domains: symptoms (assessing the frequency and severity of respiratory symptoms), activity (assessing the effects of breathlessness on mobility and physical activity), and impact (assessing the psychosocial impact of the disease). Scores are weighted such that every domain score and the total score range from 0 to 100 as per the score calculation algorithms recommended by its developer, with higher scores indicating more limitations. A mean change score of 4 units is associated with slightly efficacious treatment, 8 units for moderately efficacious treatment and 12 units for very efficacious treatment.

Results: A total of 13 subjects completed the study. The median age range was 65-74 years, 69% were male vs 31% female, 69% had a diagnosis of IPF and 31% had a diagnosis of PF. End of study scores were compared to baseline scores for the various instruments. The results observed are as follows:

WHO (five) well-being index: Of the 13 subjects, 8 (61.5%) had a significant positive change, 2 (15.4%) had no change and 2 (15.4%) showed a negative change in score as compared to baseline (FIG. 1).

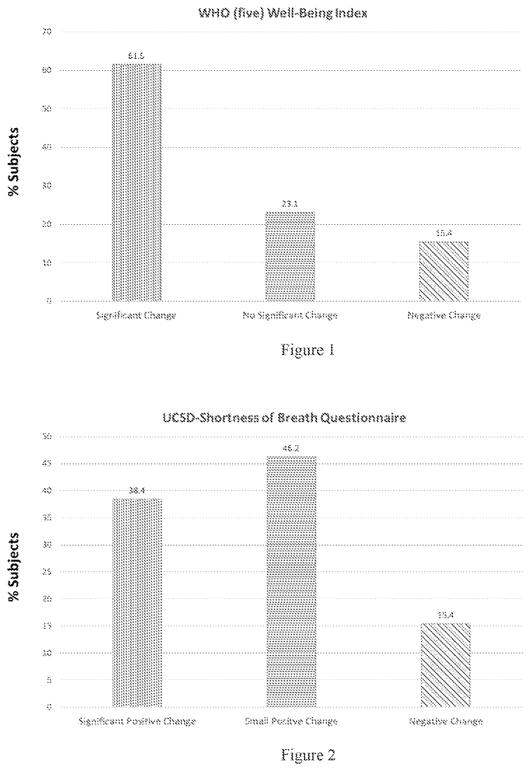

University of California San Diego Shortness-of-Breath (UCSD-SOB): Of the 13 subjects, 5 (38.4%) showed positive significant change, 6 (46.2%) showed a small positive change that was not considered a reasonable MCID, and 2 (15.4%) showed a negative change in score as compared to baseline (FIG. 2).

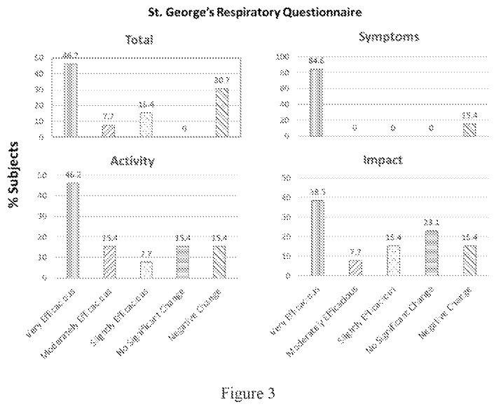

St. George's Respiratory Questionnaire (SGRQ): Of the 13 subjects, the treatment was found to be very efficacious in 6 (46.2%), moderately efficacious in 1 (7.7%), and slightly efficacious in 2 (15.4). Four (30.7%) subjects showed a decline in the total SGRQ scores. SGRQ scores were also calculated for the three domains — Symptoms, Activity and Impacts (FIG. 3) and the results are as follows:

SGRQ-Symptoms: The treatment was found to be very efficacious in 11 (84.6%) subjects. Two (15.4%) subjects showed a decline in the SGRQ symptoms score.

SGRQ-Activity: The treatment was found to be very efficacious in 6 (46.2%), moderately efficacious in 2 (15.4%), and slightly efficacious in 1 (7.7%) subject. No significant change was seen in 2 (15.4%) subjects and 2 (15.4%) subjects showed a decline in their SGRQ activity scores.

SGRQ-Impacts: The treatment was found to be very efficacious in 5 (38.5%), moderately efficacious in 1 (7.7%), and slightly efficacious in 2 (15.4%) subjects. No significant change was seen in 3 (23.1%) subjects and 2 (15.4%) subjects showed a decline in their SGRQ impacts scores.

The above data shows that supplementation with Serracor-NK and Serra RX as indicated for 3 months is beneficial in improving the feeling of well-being in PF patients.

Further, the subjects treated with Serracor-NK and Serra RX as indicated for 3 months had improved scores on the UCSD-SOBQ as compared to baseline, suggesting that supplementation with these supplements reduces shortness of breath and improves patients' ability to perform activities of daily living.

Subjects treated with Serracor-NK and Serra RX as indicated for 3 months had improved total, symptoms, activities and impacts scores on the SGRQ as compared to baseline, suggesting that treatment with these supplements improves fibrosis and related symptoms and/or reduces the frequency and severity of fibrosis and related symptoms, improves patients' ability to perform activities, enhances social and psychological health.

Improvements observed in this study are attributable to the fibrinolytic and inflammation-modulating effects of the two major systemic enzymes in the supplements—serrapeptase and nattokinase.

Conclusion: Supplementation Serracor-NK and Serra Rx improve symptoms, ability to perform activities of daily living, and mental and physical wellbeing, thus enhancing overall quality of life in patients with pulmonary fibrosis. These supplements can help alleviate symptoms and improve quality of life in pulmonary fibrosis patients.

Serracor-NK contains the fibrinolytic enzymes serrapeptase and nattokinase and other proteolytic enzymes, antioxidants and essential vitamins and minerals including bromelain, papain, lipase, rutin, amla, coenzyme Q10 and magnesium. Serra Rx is a serrapeptase supplement.

The foregoing description of the invention has been set merely to illustrate the invention and is not intended to be limiting. Since the modifications of the disclosed embodiments incorporating the spirit and substance of the invention may occur to the person skilled in the art, the invention should be construed to include everything within the scope of the disclosure.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A composition comprising the enzymes serrapeptase and nattokinase, and/or one or more additional components, for the treatment of fibrosis.

2. The composition of claim 1, wherein the one or more additional components include one or more enzymes, bioflavonoids, vitamins, coenzymes, minerals, herbs, antioxidants, probiotics, prebiotics, sugars, fibers, dietary supplements, diluents, or mixtures thereof.

3. The composition of claim 2, wherein the one or more additional components are selected from bromelain, papain, lipases, proteases, rutin, amla, coenzyme Q10, magnesium, and mixtures thereof.

4. The composition of claim 1, wherein the serrapeptase has an activity ranging from 100 SPU/g to 2 million SPU/g.

5. The composition of claim 1, wherein the nattokinase has an activity ranging from 50 FU/g to 20,000 FU/g.

6. The composition of claims 1, wherein the one or more additional components include bromelain with an activity ranging from 30 GDU/g to 3,000 GDU/g.

7. The composition of claim 1, wherein the composition has an activity ranging from 500 FU/g to 50,000 FU/g.

8. The composition of claim 1, wherein the one or more additional components are present in an amount ranging from 1 mg to 950 mg per gram.

9. A delivery system for treating fibrosis comprising:
the composition of claim 1; and
a delivery system using an enteric coating.

10. The composition of claim 1, wherein the fibrosis is idiopathic pulmonary fibrosis.

11. A method for improving fibrosis and one or more related symptoms and/or reducing the frequency and severity of fibrosis and one or more related symptoms in a subject having afibrotic disease, the method comprising administration of the composition of claim 1 to the subject.

12. A method for improving oxygen saturation, vigor, lung function, cough, phlegm, fatigue, body pain, chest discomfort, anxiety, depression, loss of appetite, respiratory disorders and/or alleviating dyspnea, exertional dyspnea, limitations and fear caused by dyspnea or combinations thereof in a subject, the method comprising administration of the composition of claim 1 to the subject.

13. A method for improving immune health by combating the oxidative stress and inflammation in animals, including humans, the method comprising administration of the composition of claim 1.

14. The composition of claim 1, wherein the composition is in a dosage form administered orally

15. The composition of claim 14, wherein the dosage form is a tablet, capsule, powder, liquid solution, liquid suspension, granules, effervescent, or oral dispersible film.

16. The method of claim 11, wherein the one or more additional components include one or more enzymes, bioflavonoids, vitamins, coenzymes, minerals, herbs, antioxidants, probiotics, prebiotics, sugars, fibers, dietary supplements, diluents, or mixtures thereof.

17. The method of claim 16, wherein the one or more additional components are selected from bromelain, papain, lipases, proteases, rutin, amla, coenzyme Q10, magnesium, and mixtures thereof.

18. The method of claim 11, wherein the serrapeptase has an activity ranging from 100 SPU/g to 2 million SPU/g.

19. The method of claim 11, wherein the nattokinase has an activity ranging from 50 FU/g to 20,000 FU/g.

20. The method of claim 11, wherein the one or more additional components include a bromelain enzyme with an activity ranging from 30 GDU/g to 3,000 GDU/g.
